(19) United States
(12) Patent Application Publication
(10) Pub. No.: US 2023/0141985 A1
(43) Pub. Date: May 11, 2023

(54) PEROXIREDOXIN 6 OR A SYNTHETIC ANALOGUE THEREOF FOR USE AS A HYPOGLYCAEMIC AGENT

(71) Applicant: Università degli Studi di Roma "Tor Vergata"

(72) Inventors: Davide LAURO, Roma (IT); Paola ROGLIANI, Roma (IT); David DELLA MORTE, Roma (IT); Barbara CAPUANI, Roma (IT); Francesca PACIFICI, Roma (IT)

(73) Assignee: Università degli Studi di Roma "Tor Vergata", Roma (IT)

(21) Appl. No.: 17/911,148

(22) PCT Filed: Mar. 12, 2021
(86) PCT No.: PCT/IT2021/050061

§ 371 Date: Sep. 13, 2022

(30) Foreign Application Priority Data
Mar. 13, 2020 (IT) 102020000005467

(51) Int. Cl. A61K 38/44; A61K 31/155; A61K 38/28; A61P 3/10; A61K 45/06

(52) U.S. Cl. CPC A61K 38/44; A61K 31/155; A61K 38/28; A61K 45/06; A61P 3/10; C12Y 111/01015

(57) ABSTRACT

The present invention relates to peroxiredoxin 6 or a synthetic analogue thereof for use as a hypoglycaemic agent, for example in the treatment of diabetes, such as type 1 diabetes mellitus and type 2 diabetes mellitus.

The present invention relates to peroxiredoxin 6 or a modified synthetic analogue thereof for use as a hypoglycaemic agent.

In particular, the invention relates to peroxiredoxin 6 or a modified synthetic analogue thereof for use as a hypoglycaemic agent, insulin secretagogue, insulin sensitiser and for the reduction of insulin resistance, for example in the treatment of type 2 diabetes mellitus or in obesity in the presence or absence of type 2 diabetes mellitus.

Diabetes mellitus (DM) is a paradigmatic example of a non-communicable chronic disease in which there is no presence of a transmissible pathogenic agent, but whose proportions, in terms of the number of people affected by the pathology, are comparable to those of a pandemic. In fact, among non-communicable chronic diseases, DM was recognised as the first pandemic by the UN (United Nations) and the WHO (World Health Organization). In DM there is always a reduction in glucose-stimulated insulin secretion by pancreatic beta cells, which is caused by a reduction in the volume and/or functionality of the pancreatic beta cells themselves. DM can be defined as a complex, heterogeneous group of chronic metabolic diseases, whose main characteristic is hyperglycaemia. DM is a multifactorial disease with different factors, both genetic and environmental, predisposing to the onset of the pathology itself. The two principal and most common forms are Type 1 diabetes mellitus (DMT1) and Type 2 diabetes mellitus (DMT2). This pathology can be caused by a prevalently autoimmune aetiology, as in DMT1, or a multifactorial one characterised by a relative reduction in insulin secretion and an increase in the levels of insulin resistance, as in DMT2. In this condition one observes increased blood insulin levels, which are insufficient, however, to overcome the high levels of peripheral insulin resistance at the level of the organs that are the target of insulin action - this in the initial stage of the pathology. An increase in the levels of insulin resistance is mainly found in overweight or obese people and/or those with a reduced physical activity. Therefore, DMT2 is a progressive metabolic disease characterised by insulin resistance and a reduction of a functional mass of pancreatic beta cells, which brings about a decrease in insulin secretion in response to glucose. In the presence of a genetic predisposition, this can cause the onset of DMT2 with hyperglycaemia, diabetic dyslipidaemia, a pro-inflammatory state, adiposopathy and an increase in the levels of oxidative stress. DMT1 manifests itself mainly in adolescent individuals and in any case a diagnosis is generally made within 30 years of age. DMT1 arises due to the destruction of insulin-producing pancreatic beta cells, which results in an almost absent secretion of insulin and an absolute need for an exogenous insulin treatment. The increase in the prevalence and incidence of DM is mainly generated by new cases of DMT2. This clinical condition is associated with an increase in the levels of insulin resistance due to excess weight, obesity and a sedentary lifestyle, which are constantly on the rise in the world population. Both of the main forms of DM can include different stages of disease, in particular DMT2, in which there is a stage of the pathology in which insulin is not necessary for the treatment of the pathology, and a stage in which insulin is required for the person’s very survival.

The third most common form of DM is gestational diabetes, which occurs in women during pregnancy. There are also other rare forms of DM, such as genetic ones or forms secondary to other pathologies.

A diagnosis of DM, with the exception of gestational diabetes, is made upon a finding, after fasting, in at least two different assays, of blood glucose levels ≥ 126 mg/dl, or blood glucose levels 2 hours after oral intake of anhydrous glucose (75 g) ≥ 200 mg/dl, random glycaemia values, i.e. independent of the intake of food, ≥ 200 mg/dl or glycosylated haemoglobin values ≥ 6.5% (48 mmol/mol).

The therapy for diabetes mellitus varies considerably according to the type of DM to be treated, type 1 or type 2, and in DMT2 it can differ further according to the stage of the disease. For example, the treatment of DMT2 can be different in the initial phase, in which there is a partial loss of pancreatic beta cell function, or in the advanced stage of the pathology in the case of long-term disease, in which there is a major failure of the endocrine pancreas. In DMT1 the main clinical sign is the absolute absence or a nearly complete loss of pancreatic beta cell function, and hence of insulin secretion in response to glucose. In this case treatment with insulin is fundamental for all people with this pathology and, therefore, an insulin replacement therapy must be proposed. In addition to hyperglycaemia, hypoinsulinaemia can contribute to other metabolic disorders such as hypertriglyceridaemia, ketoacidosis and the onset of a catabolic state which can be life-threatening. In the past thirty years, evidence has been gathered in favour of an intensive insulin treatment with multiple daily injections or with continuous subcutaneous insulin infusion by means of a pump. The Diabetes Control and Complications Trial (DCCT) (The Diabetes Control and Complications Trial Research Group. The effect of intensive treatment of diabetes on the development and progression of long-term complications in insulin-dependent diabetes mellitus. N Engl J Med 1993;329: 977-986) demonstrated that good metabolic control was obtained with intensive insulin treatment, resulting in a reduction in long-term chronic complications. The therapy for DMT2 is different from that for DMT1, since the deficiency of insulin secretion is partial and there are different etiopathogenetic defects that contribute to determining the onset of the pathology. These include a) a reduction in glucose-dependent insulin secretion; b) an increase in the secretion of glucagon by pancreatic alpha cells; c) an increase in hepatic glucose production; d) a dysfunction of neurotransmitters and an increase in insulin resistance in the brain; e) an increase of lipolysis; f) an increase in renal glucose reabsorption; g) a reduction in the incretin effect of intestinal ‘incretin’ hormones; h) a reduction in glucose uptake in peripheral tissues such as skeletal muscle, the liver and adipose tissue.

The main mechanism of action of the presently available therapies for reducing blood glucose levels consists in the treatment of one or more of these alterations. Furthermore, the factors determining which hypoglycaemic therapy is suitable for the treatment of a specific patient differ and the main ones are: 1) the presence of major comorbidities and/or chronic complications such as atherosclerosis-based cardiovascular disease, or an increase in the risk factors that determine it, diabetic nephropathy, microvascular complications, etc.; 2) the risk of hypoglycaemia; 3) the effects on body weight; 4) side effects; 5) cost; and 6) patient preferences. It is important to underscore, together with the pharmacological treatment, the importance of changes in lifestyle designed to reduce body weight and increase physical activity. Metformin, a biguanide, is generally the first drug for the treatment of DMT2, in all age groups. Metformin increases uptake and reduces hepatic glucose production, gluconeogenesis, again at the hepatic level, by acting mainly on the mitochondria, thereby increasing the insulin sensitivity of tissues sensitive to the action of insulin. Therefore, the levels of insulin resistance, the blood concentrations of lipids and the desire to eat food are reduced, which can also bring about a modest reduction in body weight. The undesirable effects are above all at a gastrointestinal level with bloating, abdominal discomfort and diarrhoea; phenomena associated with the onset of diabetic ketoacidosis may occur more rarely, in particular in people with severe kidney failure and kidney filtration levels < a 30 mL/min/1,73 m². If the control of glycaemia is not optimal and the values of glycosylated haemoglobin are greater than or equal to 1.5% or 12.5 mmol/mol, above the glycaemic targets treatment with a second hypoglycaemic drug or basal insulin (with long-lasting action) should begin. In patients with DMT2, after several years, about 7 years according to the estimates of the UKPDS (P D Home “Impact of the UKPDS--an Overview” Diabet Med, 25 Suppl 2, 2-8 Aug. 2008), the monotherapy will fail and there will be a need to use a second hypoglycaemic drug. There exist different therapeutic options according to the clinical characteristics of the patient, such as the presence of chronic complications like cardiovascular disease, diabetic retinopathy, diabetic nephropathy, neuropathy, etc., and the presence of other concomitant pathologies such as hypertension, etc. Furthermore, the different therapeutic options also have to take into account the social aspects and therapeutic targets that it is desired to reach, while reducing to a minimum the risk of side effects such as hypoglycaemia and noncompliance with the treatment. Incretin mimetic drugs are a valid option as hypoglycaemic drugs that can be associated with the treatment with metformin. These drugs act to restore the incretin effect, which, stated briefly, determines about 70% of insulin secretion in response to an oral glucose load. Therefore, these drugs enhance glucose-dependent insulin secretion, i.e. in the presence of increased blood glucose levels, such as after a meal, and reduce glucagon secretion. The two incretin hormones that modulate glycaemic control are the glucose-dependent insulinotropic peptide (GIP) and the glucagon-like peptide (GLP-1). These peptides have a brief half-life, since they are hydrolysed in a few minutes by dipeptidyl-peptidase-4 (DPP-4). In patients with DMT2, the incretin effect mediated by these hormones is reduced or absent. There exist 2 classes of hypoglycaemic drugs that act to improve this defect and they are GLP-1 receptor agonists (GLP-1 RA) and dipeptidyl-peptidase-4 inhibitors (DPP-4i). In people with DMT2, this treatment improves glycaemic control, also reducing body weight (in particular GLP-1 RA) and systolic pressure. The risk of hypoglycaemia is low (except when the treatment is in association with a sulfonylurea), because of their glucose-dependent mechanism of action. GLP-1 RAs have recently demonstrated a protection against cardiovascular disease and kidney disease in the DMT2 population. Therefore, these drugs can be indicated for a large part of the DMT2 population and their effectiveness has recently also been demonstrated in primary cardiovascular prevention in the DMT2 population. Different pleiotropic effects independent of the hypoglycaemic action have been proposed and some of them demonstrated. The second class of drugs which act on the incretin system are the DPP-4is. These drugs have a more modest effect in controlling hyperglycaemia compared to the GLP-1 RAs, but they can also reduce the levels of postprandial hypertriglyceridaemia. The undesirable effects are minimal and the main action is that of inhibiting DPP-4, whose activity increases in people with DMT2, and restoring physiological concentrations of GLP-1, which are normally decreased in DMT2. This brings about an improvement in pancreatic beta cell function and a reduction in blood glucose levels. There are few undesirable effects and the risk of hypoglycaemia is present when the therapy is used together with sulfonylureas or insulin.

Sulfonylureas are an old class of oral hypoglycaemic drugs that increase insulin secretion independently of blood glucose levels. They perform their action by blocking ATP-dependent potassium (K_{ATP}) channels and reducing hepatic gluconeogenesis; however, sulfonylureas entail an increased risk of hypoglycaemia compared to more recent hypoglycaemic drugs. Nowadays, sulfonylureas are rarely recommended and are proposed as third-line drugs, to be used in specific types of patients. Meglitinides, of which only repaglinide is commercially available in Italy at present, are insulin secretagogues that act like sulfonylureas on pancreatic beta cells, but with a lower binding affinity, and they can thus be considered sulfonylureas with a reduced half-life and more manageably used. In association with metformin or in a triple hypoglycaemic therapy use can be made of thiazolidinediones, of which only pioglitazone is commercially available at present. Thiazolidinediones are peroxisome proliferator-associated receptor y (PPAR y) agonists which increase peripheral insulin sensitivity and glucose uptake by skeletal muscle fibre, the liver and adipose tissue. Furthermore, they can decrease tissue accumulation of triglycerides and the secretion of pro-inflammatory cytokines, whose levels are increased in DMT2, thus better preserving the function and integrity of pancreatic beta cells. It should be noted that in any case the functionality and mass of pancreatic beta cells is lost over time, as there is an increase both in cell death by apoptosis, and in the dedifferentiation of pancreatic beta cells towards a less differentiated phenotype. At present there exists only one class of hypoglycaemic drugs that act by reducing the levels of glycaemia independently of the action and secretion of insulin, namely SGLT-2 inhibitors, which perform their action at the renal level by inhibiting the sodium glucose cotransporter-2 (SGLT-2) of the renal proximal tubule and increasing the excretion of glucose (glycosuric effect) and sodium in urine. This brings about a reduction in blood glucose levels and, together with other pleiotropic effects that have not yet been fully clarified, it has important effects on various other parameters (reduction in arterial pressure, in body weight, etc.). This class of drugs brings about a reduction in the risk of cardiovascular disease and of progression of kidney disease. In particular, there is a reduction in the risk of occurrence of heart failure or an improvement in the latter if present.

Insulin therapy is recommended in a person with DMT2 in the presence of a catabolic state characterised by weight loss, hypertriglyceridaemia and ketosis. Furthermore, if the levels of glycaemia are particularly high, with values of glycosylated haemoglobin exceeding 10% (86 mmol/mol) or values of fasting glycaemia greater than or equal to 300 mg/dl, insulin treatment is recommended.

What has been described above reveals that there are still unmet needs as regards the treatment of DMT1 and DMT2 with hypoglycaemic drugs. In particular, no treatment is yet available which can restore the function of pancreatic beta cells or reduce the failure thereof. A further problem yet to be resolved is the need to use, during the clinical progression of DMT2, several hypoglycaemic drugs which act on different pathophysiological defects in order to achieve good metabolic compensation. Furthermore, it is important to find drugs with a reduction of the side effects present in the treatments outlined above.

In the light of what has been described, it appears evident that there is a need to have new therapies for the treatment of diabetes, such as DMT2, which overcome the disadvantages of the known therapies.

Peroxiredoxin 6 (Prdx6) is a peptidic antioxidant enzyme, discovered in 1998, which belongs to the peroxiredoxin family (Prdx 1-6). In particular, the enzyme Prdx6 has a length of 224 amino acids and a mass of 25,035 Da. To date, six molecules of the peroxiredoxin family (Prdxs) have been identified. These molecules are of great interest, because, besides their capacity to neutralise a vast array of reactive oxygen species (ROS), they can play other important roles, acting as chaperone molecules, intracellular signal regulators and regulatory molecules.

Three main activities have been described up to now for Prdx6: 1) GSH (glutathione) peroxidase-dependent activity (GPx) (Cys47); 2) phospholipase-A₂ calcium-independent activity (GXSXG sequence 30-34); and 3) a specific lysophosphatidylcholine acyltransferase activity which enables, among other actions, a reduction in phospholipid hydroperoxide with the electrons donated by glutathione. Furthermore, through these actions Prdx6 can also regulate the transmission of cellular signals by modulating different pathways thereof. This molecule was identified in blood, even though its origin has not yet been defined, and is presently considered a potential biomarker of various metabolic diseases, such as DMT2.

In fact, in people with DMT2, the levels of Prdx6 are higher than in healthy controls (Al-Masri AA et al. Differential associations of circulating peroxiredoxins levels with indicators of glycemic control in type 2 diabetes mellitus Eur Rev Med Pharmacol Sci. 2014;18(5):710-6). The blood concentrations of Prdx6 are negatively correlated with those of glycosylated haemoglobin and fasting glycaemia. Patients with poor control of glycaemia have reduced values of Prdx6, compared to patients with good glycaemic control (E. El Eter and A.A. Al-Masri Peroxiredoxin isoforms are associated with cardiovascular risk factors in type 2 diabetes mellitus Braz J Med Biol Res. 2015 May; 48(5): 465-469). Furthermore, Prdx6 is positively correlated with blood insulin concentrations, which is a specific and peculiar characteristic of Prdx6 (Al-Masri AA et al. Differential associations of circulating peroxiredoxins levels with indicators of glycemic control in type 2 diabetes mellitus Eur Rev Med Pharmacol Sci. 2014;18(5):710-6).

Recently, studies conducted on mouse models lacking the peroxiredoxin 6 (Prdx6^{-/-}) gene showed that the absence of this antioxidant enzyme determines a reduction in glucose-stimulated insulin secretion (GSIS) and increases the levels of insulin resistance in skeletal muscle fibre (Pacifici F, et al. Peroxiredoxin 6, a novel player in the pathogenesis of diabetes. Diabetes. 2014;63(10):3210-20).

It is known, moreover, that Prdx6 can stimulate insulin secretion by insulinoma cells (RIN-m5F β) (Novoselova EG et al. Protective Effect of Peroxiredoxin 6 Against Toxic Effects of Glucose and Cytokines in Pancreatic RIN-m5F β-Cells. Biochemistry (Mosc). 2019;84(6):637-643). However, these results do not provide any information on a possible therapeutic application of the molecule in diabetes mellitus. In particular, the authors conducted a study in which they showed that there is a protective action of Prdx6 in the modulation of apoptosis induced by oxidative stress in response to treatment with pro-inflammatory cytokines and hyperglycaemia in tumour cells. Furthermore, the authors described that Prdx6 can stimulate insulin secretion and hypothesised that this effect is mediated by modulation of the activation of NF-_{K}B, i.e. through one of the classic mechanisms tied to the specific antioxidant action of this enzyme and not through a direct effect of Prdx6 in stimulating insulin secretion.

According to the present invention, it has been demonstrated that Prdx6 can stimulate insulin secretion in human islets of Langerhans and induce the secretion of GLP-1 (glucagon like peptide-1), with a receptorial mechanism of action. Therefore, the present invention has demonstrated, for the very first time, the stimulation of insulin secretion both directly by Prdx6 and indirectly by GLP-1, an effect, in any case, that is not mediated by the classic actions of this enzyme. This mechanism of action would be particularly effective in the treatment of Type 2 diabetes mellitus.

The results obtained according to the present invention show that the insulin secretion stimulated by Prdx6 is independent of the action of other agents that induce insulin secretion. Furthermore, on the basis of the results according to the present invention, Prdx6 increases the levels of insulin secretion stimulated by glucose, even though this increase does not reach statistical significance.

The experimental results also show that Prdx6 can induce the secretion of the incretin hormone GLP-1 in NCI-H716 intestinal cells, cells similar to the L cells of the ileum/colon. Thanks to the induction of the secretion of GLP-1 there would be a specific effect for DMT2. Therefore, Prdx6 shows a “hormone-like” action, acting on several peripheral organs. As a further confirmation of this, the peptidic tertiary and quaternary structure of the molecule shows a similarity with that of some peptide hormones. The experimental results demonstrate that Prdx6 can be advantageously used in the therapy of DMT2 as a GLP-1 secretion-stimulating drug, as the blood concentrations of GLP-1 are reduced, above all in the postprandial period, in patients with DMT2. By restoring the secretion of GLP-1, which acts on pancreatic beta cells, Prdx6 can increase glucose-stimulated insulin secretion. In fact, GLP-1 increases glucose-stimulated insulin secretion, as stated above. Therefore, a restoration of the incretin effect would be obtained.

In particular, in the examples that follow, it is shown that NCI-H716 cells (human colorectal adenocarcinoma cells) differentiated towards an endocrine phenotype, similar to that of the L cells of the ileum/colon, and stimulated with 400 nM of Prdx6 recombined individually or with 25 mM glucose at 0, 15′, 30′, 60′ and 120′ min. significantly increase the secretion of GLP-1 , in particular after 15 min. and 60 min.

According to the present invention, it has been further observed that Prdx6 stimulates insulin secretion independently of the concentrations of glucose and that the effect of Prdx6 is not added to that of glucose, but can be synergic. Therefore, treatment with Prdx6 induces insulin secretion independently of the stimulation with glucose and glucose-stimulated insulin secretion increases in the presence of Prdx6, even if not to a statistically significant degree. The effect of Prdx6 in inducing glucose-stimulated insulin secretion can thus be considered synergic. However, the effect cannot be considered additive, since the levels of insulin secretion in response to the co-treatment with Prdx6 and glucose are significantly lower than those present in response to treatment with Prdx6 alone.

Furthermore, together with the action of glucose-independent stimulation of insulin secretion (independent of the concentrations of glucose), according to the present invention, a mechanism of regulation of glucose-stimulated insulin secretion mediated by the increase in GLP-1 secretion in response to Prdx6 has been observed.

Therefore, Prdx6 can be advantageously used as a hypoglycaemic agent in people with DMT2 and in DMT1 through the stimulation of insulin secretion in vivo.

According to the present invention, the data obtained show that Prdx6 regulates insulin secretion by means of a glucose-independent mechanism (independently of the concentrations of glucose), increases glucose-dependent insulin secretion and also stimulates the secretion of GLP-1.

In addition to being an insulin secretagogue agent, Prdx6 also has the advantage of reducing the cell death of pancreatic beta cells by virtue of its documented anti-apoptotic effect, obtained by reducing the cellular oxidative stress levels (reduction in the cellular concentrations of ROS -Reactive Oxidative Species).

Therefore, according to the present invention Prdx6 can be advantageously used in therapy for DMT2 and DMT1 to improve the homeostasis of glucose, reduce hyperglycaemia and preserve the pancreatic beta cell functional mass.

According to the experimental results reported further below, Prdx6 is capable of significantly decreasing the process leading to the reduction of the pancreatic beta cell functional mass under conditions of increased oxidative stress as in DMT2, DMT1 and obesity, in particular obesity in DMT1 and DMT2 patients.

Finally, it may be deduced from the preclinical data (in vitro and ex-vivo) shown below that Prdx6 can preserve and conserve pancreatic beta cell function in patients with DMT2 and DMT1 by reducing or inhibiting the progressive decrease of the pancreatic beta cell functional mass.

The use of Prdx6 as a hypoglycaemic drug according to the present invention could reduce the number of patients who pass from a treatment with oral or injective hypoglycaemic drugs towards treatment with insulin by decreasing the progression of pancreatic beta cell dysfunction.

It is therefore specific object of the present invention peroxiredoxin 6 (Prdx6) (natural or synthetic) or a synthetic analogue thereof for use as a hypoglycaemic drug, Prdx6 having activity both as an insulin secretagogue and an insulin sensitiser, i.e. being a molecule that can act both on insulin secretion and on insulin sensitivity.

In particular, the present patent application concerns peroxiredoxin 6 or a synthetic analogue thereof for use in the treatment of diabetes mellitus, such as type 2 diabetes mellitus and type 1 diabetes mellitus, pre-diabetes and complications of diabetes mellitus, for example microvascular and macrovascular pathologies such as diabetic retinopathy, diabetic neuropathy, peripheral and cardiac diabetic vasculopathy.

According to one aspect of the present invention, the uses of peroxiredoxin 6 or of a synthetic analogue thereof, according to the present invention, can be aimed at a population of patients affected by obesity, in particular obesity in DMT1 and DMT2 patients.

According to the present invention, said synthetic analogue thereof can be a synthetic analogue of the native protein (peroxiredoxin 6) or a synthetic analogue modified in one or more catalytic domains selected from the peroxidase site, phospholipase-A₂ site, lysophosphatidylcholine acyltransferase site, dimerization site and sumoylation site. Preferably, the dimerization site can be inhibited to obtain the protein in monomeric form.

For example, said synthetic analogue thereof can be selected among the following mutant Prdx6s: mutant inhibited in the dimerization site, such as L145E, L148E or L145E/L148E, mutant inhibited in the phospholipase-A₂ site, such as S32A or S32T, mutant inhibited in the peroxidase site, such as C47S, mutant inhibited in the sumoylation site, such as K122R, K142R or K122R/K142R.

Table 1 sums up the examples of the above-mentioned mutant Prdx6s.

TABLE 1

| | | | | |
|---|---|---|---|---|
| Mutant Prdx6 | Inhibition of dimerization site | Inhibition of phospholipase -A₂ site | Inhibition of peroxidase site | Inhibition of sumoylation site |
| | L145E | S32A | C47S | K122R |
| | L148E | S32T | | K142R |
| | L145E/L148E | | | K122R/K142/R |

On the basis of experimental results, it is plausible that Prdx6 has a mechanism of action mediated by a membrane receptor. In particular, the experimental data described further below show that exogenous Prdx6 is localised mainly at the level of the cytoplasmic membrane (by marking of Prdx6 with biotin), suggesting an action of a receptorial type. This is further corroborated by the maximum secretion of insulin and GLP-1 after 15 minutes, which would confirm the hypothesis of a receptorial mechanism of action.

Therefore, the mechanism according to the present invention is different from the known actions of Prdx6s, which are: 1) peroxidasic, where the main substrates are H₂O₂ (hydrogen peroxide), short-chain hydroperoxides, phospholipid hydroperoxides and peroxynitrites; 2) phospholipase-A₂; and 3) lysophosphatidylcholine acyltransferase (LPCAT).

On the basis of the foregoing, the use of synthetic analogues in which there is a mutation leading to the inhibition of the peroxidasic or phospholipase-A₂ action cannot inhibit the effectiveness of Prdx6 as a hypoglycaemic agent in diabetes, whereas it is plausible that it favours the binding of Prdx6 with the membrane receptor, thereby improving the action of Prdx6 itself as a hypoglycaemic agent.

Furthermore, the (reduced) native protein is in a dimeric form structurally, both in crystals and in solution, with the monomers arranged in an anti-parallel manner. Dimerization occurs between hydrogen bonds which involve the interface between the seven filaments of the beta sheet of the secondary structure of the protein. The hydrophobic residues Leu145, lle 147, Leu 148 and Pro 150 of the two monomers help to stabilise the interface (Aron B Fisher Peroxiredoxin 6 in the repair of peroxidized cell membranes and cell signaling Arch Biochem Biophys 2017 Mar 1;617:68-83). It is believed that the presence of Prdx6 in monomeric form can render the receptor binding more efficient, thus enhancing the action of Prdx6.

Finally, Prdx6 can also be aberrantly sumoylated at the level of lysines 122 and 142 during oxidative stress. This provokes a reduction both in gene expression and in the abundance of the protein itself (Chhunchha, B.; Kubo, E.; Fatma, N.; Singh, D.P. Sumoylation-deficient Prdx6 gains protective function by amplifying enzymatic activity and stability and escapes oxidative stress-induced aberrant Sumoylation. Cell Death Dis. 2017, 8, e2525).

Therefore, it is plausible that the modification, and in particular the inhibition, of the dimerization and sumoylation sites of Prdx6 may increase the effectiveness of the action of Prdx6 at the receptor level and, therefore, increase the effect on the secretion of insulin and GLP-1.

The present invention further relates to a pharmaceutical composition comprising peroxiredoxin 6 and/or a synthetic analogue thereof (i.e. one or more analogues) as an active ingredient, together with one or more excipients and/or adjuvants, for use as a hypoglycaemic agent having activity as an insulin secretagogue, GLP-1 secretagogue and insulin sensitiser. In particular, the present invention relates to the pharmaceutical composition defined above for use in the treatment of diabetes mellitus, such as type 1 diabetes mellitus and type 2 diabetes mellitus, pre-diabetes and the complications of diabetes mellitus, for example microvascular and macrovascular pathologies such as diabetic retinopathy, diabetic neuropathy, peripheral and cardiac diabetic vasculopathy.

According to one aspect of the present invention, the uses of the pharmaceutical composition according to the present invention can be aimed at a population of patients affected by obesity, in particular DMT1 and DMT2 patients affected by obesity.

According to the present invention, said synthetic analogue thereof, possibly contained in the composition, can be a synthetic analogue of the native protein or a synthetic analogue modified in one or more catalytic domains selected from the peroxidase site, phospholipase-A₂ site, lysophosphatidylcholine acyltransferase site, dimerization site and sumoylation site. For example, said synthetic analogue thereof can be selected from the following mutant Prdx6s: mutant with the dimerization site inhibited, such as L145E, L148E or L145E/L148E, mutant with the phospholipase-A₂ site inhibited, such as S32A or S32T, mutant with the peroxidase site inhibited, such as C47S, and mutant with the sumoylation site inhibited, such as K122R, K142R or K122R/K142R.

According to the present invention, the pharmaceutical composition can further comprise one or more hypoglycaemic agents other than peroxiredoxin 6 or a synthetic analogue thereof (i.e. in addition thereto), such as metformin, GLP-1 agonists, DPP-4 inhibitors, SGLT-2 inhibitors, human insulin or slow, rapid or ultra-rapid analogue insulin.

The pharmaceutical composition according to the present invention can be in a form for subcutaneous administration.

The present invention further relates to a combination of peroxiredoxin 6 or a synthetic analogue thereof with one or more hypoglycaemic agents other than peroxiredoxin 6 or a synthetic analogue thereof, such as metformin, GLP-1 agonists, DPP-4 inhibitors, SGLT-2 inhibitors, human insulin or analogue insulin, slow, rapid or ultra-rapid, for separate or sequential use as a hypoglycaemic agent.

Separate use means the administration, at the same time, of the active ingredients of the combination according to the invention in distinct pharmaceutical forms.

Sequential use means the successive administration of the active ingredients of the combination according to the present invention, each in a distinct pharmaceutical form.

In particular, the present invention relates to the combination as defined above for separate or sequential use of the active ingredients in the treatment of diabetes mellitus, such as type 2 diabetes mellitus and type 1 diabetes mellitus, pre-diabetes and complications of diabetes mellitus, for example microvascular and macrovascular pathologies such as diabetic retinopathy, diabetic neuropathy, peripheral and cardiac diabetic vasculopathy.

According to one aspect of the present invention, the uses of the combination according to the present invention can be aimed at a population of patients affected by obesity, in particular DMT1 or DMT2 patients affected by obesity.

According to the present invention, said synthetic analogue thereof, possibly present in the combination of the invention, can be a synthetic analogue of the native protein or a synthetic analogue modified in one or more catalytic domains selected from the peroxidase site, phospholipase-A₂ site, lysophosphatidylcholine acyltransferase site, dimerization site and sumoylation site.

In particular, said synthetic analogue thereof can be selected from the following mutant Prdx6s: mutant with the dimerization site inhibited, such as L145E, L148E or L145E/L148E, mutant with the phospholipase-A₂ site inhibited, such as S32A or S32T, mutant with the peroxidase site inhibited, such as C47S, and mutant with the sumoylation site inhibited, such as K122R, K142R or K122R/K142R.

On the basis of what has been set forth above, it is evident that the present invention describes, for the very first time, the use of Prdx6 as a hypoglycaemic drug. To date, the existing knowledge, such as for example the study conducted by Novoselova et al. (Novoselova EG et al. Protective Effect of Peroxiredoxin 6 Against Toxic Effects of Glucose and Cytokines in Pancreatic RIN-m5F β-Cells. Biochemistry (Mosc). 2019;84(6):637-643), neither hypothesises nor suggests that Prdx6 can be a potential hypoglycaemic drug, also in consideration of the fact that no action of Prdx6 via a specific receptor has ever been described or hypothesised. According to the present invention, by contrast, a potential action of Prdx6 mediated by a membrane receptor mechanism has been shown. Furthermore, the studies of Novoselova et al. were not conducted on the basis of analyses on islets of animal or human derivation, but were rather conducted using insulinoma cells, which are not a suitable or sufficient means for evaluating the action of potential insulin secretagogues.

The present invention will now be described by way of non-limiting illustration according to a preferred embodiment thereof, with particular reference to the examples and the figures in the appended drawings, wherein:

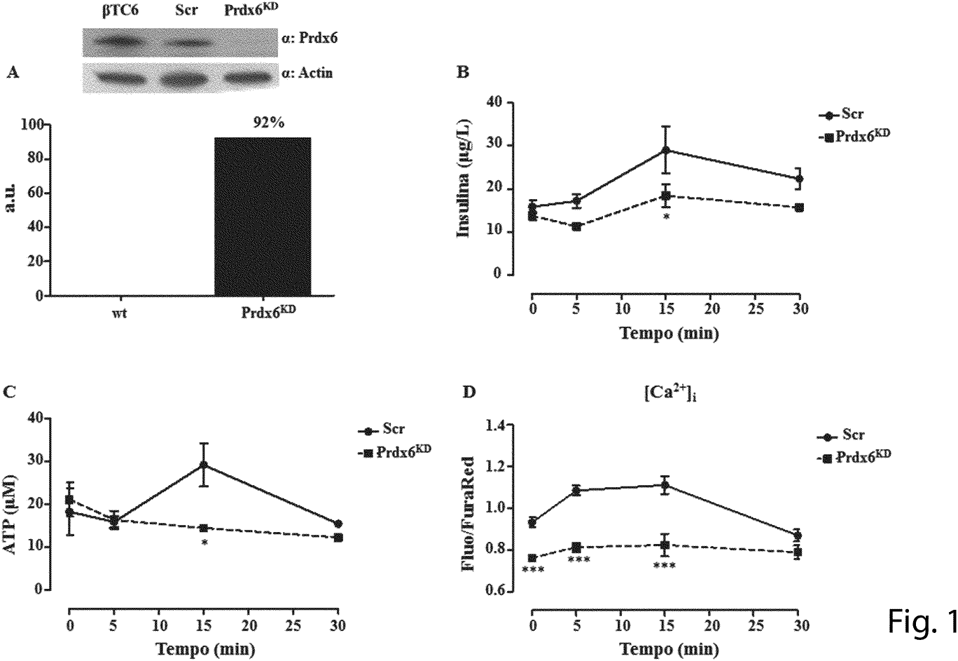

- FIG. 1 shows the deletion of Prdx6 which alters insulin secretion by modulating the synthesis of ATP and the intracellular content of Ca²⁺; A) Stable silencing of Prdx6 (Prdx6^{KD}) and respective control cells silenced for the gene encoding GFP (green fluorescent protein) (Scramble, Scr), in murine insulinoma cells βTC6; B) Evaluation of insulin secretion after stimulation with 20 mM of glucose at different time intervals (0-5-10-15-20-25-30 min.); C) the production of ATP was measured under the same experimental conditions as described in point B; D) the intracellular calcium content was analysed with a cytofluorometric assay under the same experimental conditions as used for the evaluation of insulin secretion; (*p<0.05; ***p<0.0005);

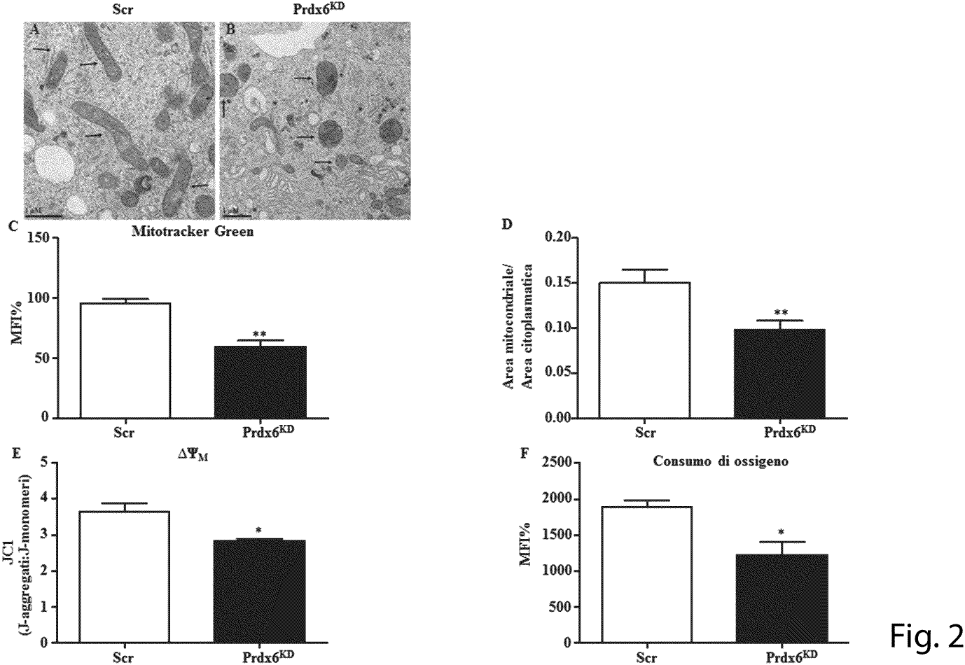

- FIG. 2 shows the structural and functional alterations of mitochondria in Prdx6^{KD} cells; A-B) Evaluation of the mitochondrial ultrastructure in Scr and Prdx6^{KD} cells through an analysis with electron microscopy; C) the mitochondrial mass was measured by flow cytofluorometry analysis using the marker MitoTracker green; D) Ratio between mitochondrial area and cytoplasmic area. E-F) Evaluation of mitochondrial functionality with an analysis of the levels of the membrane potential and oxygen consumption; (*p<0.05; **p<0.005);

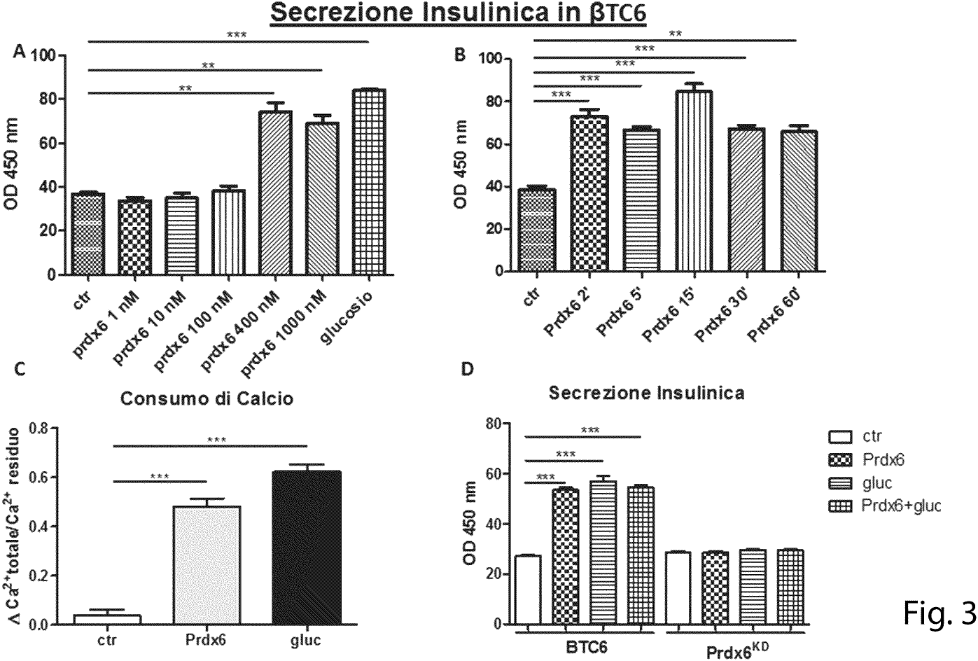

- FIG. 3 shows the evaluation of insulin secretion and calcium (Ca²⁺) levels: A-B) insulin secretion was evaluated in murine insulinoma cells βTC6) first following stimulation with different concentrations of recombinant Prdx6 for 15 minutes, and then with Prdx6 400 nM at different time intervals; C) The analysis of calcium levels used during stimulation with Prdx6 400 nM for 15 min., in association with increased levels of insulin secretion, reveals a calcium-mediated Prdx6 secretion mechanism; D) Analysis of the insulin secretion in βTC6 cells and in βTC6 cells silenced for Prdx6 (Prdx6^{KD}); the Prdx6^{KD} cells that show morphological-structural alterations of the mitochondria, responsible for the insulin secretion process, are not capable of responding to stimulation with Prdx6; (**p<0.005; ***p<0.0005);

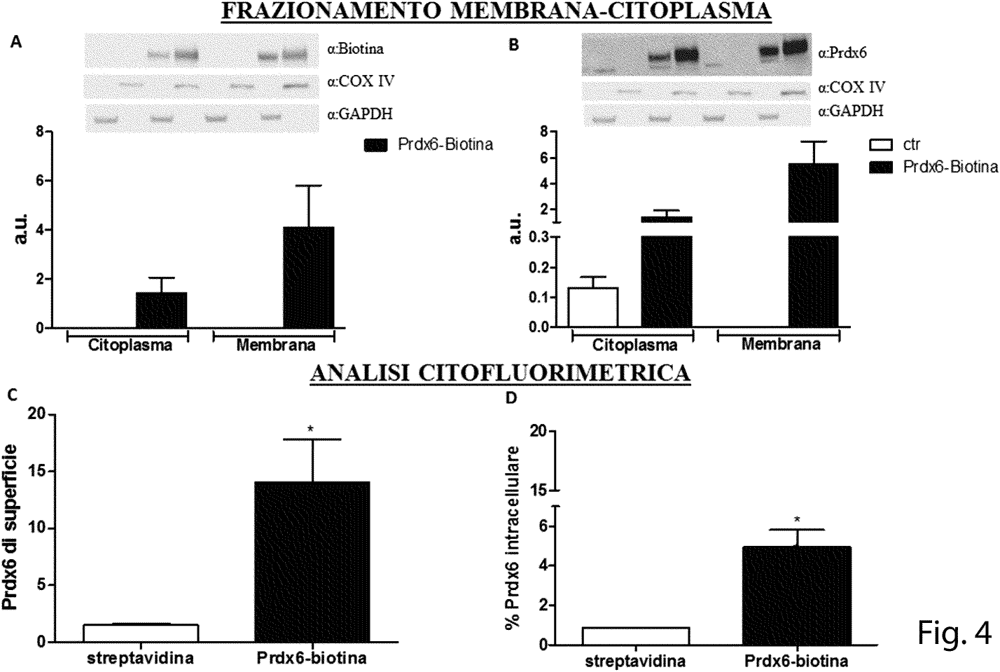

- FIG. 4 shows the localisation of the Prdx6-biotin complex; for the purpose of localising Prdx6, the latter was associated with biotin by cross-linking and the Prdx6-biotin complex was used for treatment of the cells A-B) Western blot analysis of the membrane fractionation reveals a localisation of the complex in question mainly at the level of the cytoplasmic membrane; C-D) Flow cytofluorometry analysis for the evaluation and validation of the localisation of the Prdx6-biotin complex through the use of streptavidin, which recognises and binds biotin; (*p<0.05);

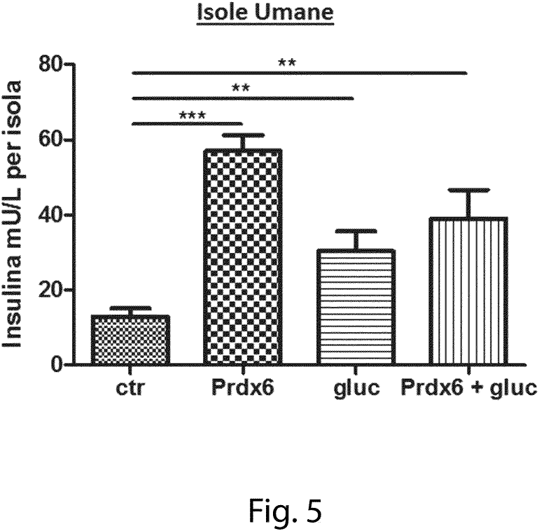

- FIG. 5 shows the Prdx6-mediated insulin secretion in human islets; Islets of healthy donors were treated with Prdx6 400 nM for 15 min.; the insulin secretion was subsequently evaluated, (**p<0.005; ***p<0.0005) (N ₌ 6);

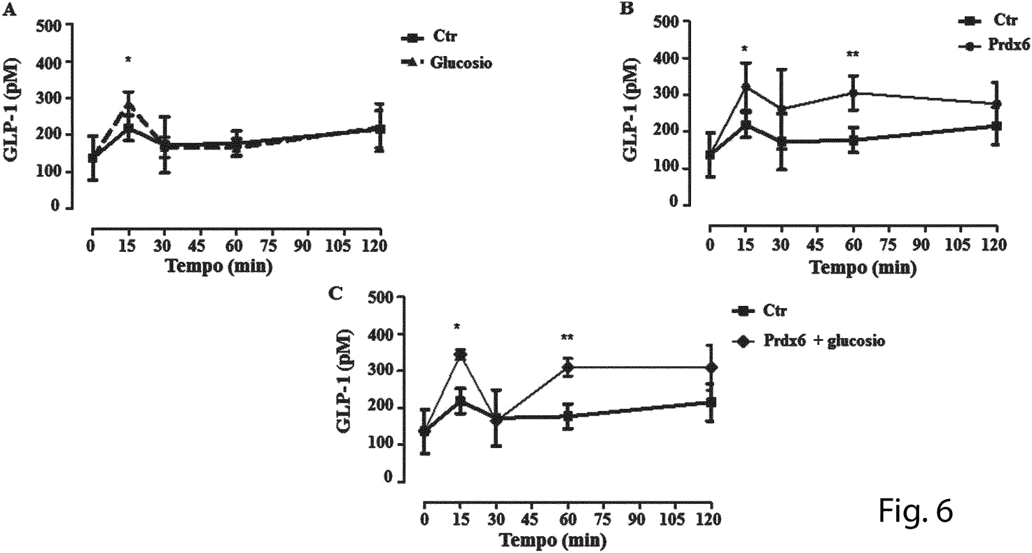

- FIG. 6 shows the Prdx6-mediated incretin secretion in human colorectal adenocarcinoma cells; colorectal adenocarcinoma cells were treated with glucose (A), Prdx6 (B), and Prdx6 + glucose (C); stimulation with Prdx6 is capable of inducing the release of GLP-1; (*p<0.05; **p<0.005);

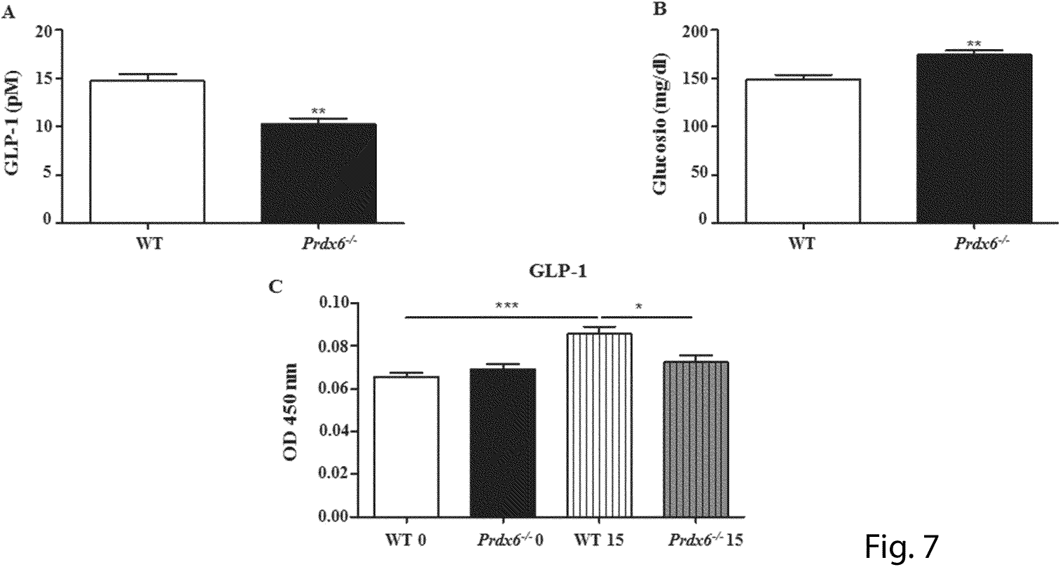

- FIG. 7 shows the incretin secretion in Prdx6^{-/-} mouse models; A-B) Prdx6^{-/-} mouse models were fed regularly and in the postprandial phases both the secretion of GLP-1 and the glycaemic levels were evaluated; subsequently, to validate the data, the animals were subjected to oral administration of a glucose load (OGTT) C). The secretion of GLP-1 is decreased in the absence of Prdx6. *p<0.05; **p<0.005; ***p<0.0005; (N = 5).

### EXAMPLE1 EXPERIMENTAL DESIGN AND RESULTS: PRDX6 AND INSULIN

The mechanism whereby Prdx6 modulates glucose-dependent insulin secretion was studied in vitro using murine insulinoma cells (βTC6) stably silenced for Prdx6 (Prdx6^{KD}) ([3TC6: ATCC-CRL-11506, Manassas, Virginia, USA) (FIG. 1, Panel A).

The cells were plated in a 24-well multiwell plate for 24 hours and were then incubated in a Krebs solution (20% Solution A: 610 mM NaCI, 24 mM KCI, 6 mM KH₂PO₄, 6 mM MgSO₄-7H₂O, 5 mM CaCl₂; 25% Solution B: 10 mM HEPES, 20 mM NaHCO₃, 20 mM NaCI, 55% H₂O and 2 mM Glucose) for 45 minutes (starvation). At the end of starvation, the culture medium was removed and a Krebs solution containing 20 mM glucose was added for 0, 5′, 15′ and 30′ minutes (FIG. 1, Panel B). The data obtained confirmed what had previously been observed in vivo: 15 minutes after stimulation with glucose, the secretion of insulin showed to be significantly reduced in the Prdx6^{KD} cells (FIG. 1, Panel B). Furthermore, under the same experimental conditions, a significant reduction in the synthesis of ATP was present in the Prdx6^{KD} cells (FIG. 1, Panel C) and, consequently, a reduced intracellular flow of Ca²⁺ (FIG. 1, Panel D).

These data thus showed a direct involvement of Prdx6 in the ‘classic’ insulin secretion process. The morphology and structure of the mitochondria were subsequently analysed by electron microscopy (FIG. 2, Panels A and B) and a significant morphological-structural alteration in Prdx6^{KD} cells was documented. Furthermore, both the mass and the dimensions of the mitochondria were evaluated, revealing a reduction in Prdx6^{KD} cells (FIG. 2, Panels C and D respectively). In association with the morphological analysis, mitochondrial functionality was also evaluated. In particular, a reduction was observed both in the mitochondrial membrane potential and in oxygen consumption in the absence of Prdx6 (FIG. 2, Panels E and F respectively). These data reveal, at least in part, the cause that determines the reduced insulin secretion in response to glucose. Subsequently, the effect of Prdx6 in stimulating insulin secretion was evaluated hypothesising a hormone-like action of Prdx6, on the basis of the results obtained. For this purpose, the murine insulinoma-βTC6 control cells and the Prdx6^{KD} cells were treated with Krebs as previously described and, after the starvation process, Krebs containing increasing concentrations of recombinant Prxd6 (1, 10, 100, 400, 1000 nM) was added for 15 minutes. The supernatant was then recovered and used to measure the insulin by means of the ELISA assay (Mercodia). The largest increase in insulin secretion, which was comparable to the one obtained after stimulation with 20 mM glucose, occurred after the treatment with 400 nM Prdx6 (p<0.0064), whereas at higher concentrations (1000 nM) it was not possible to observe a further increase in insulin secretion (FIG. 3, Panel A). Subsequently, a time response curve (0, 2, 10, 15, 30, 60 minutes) was derived by using 400 nM of Prdx6 with the aim of validating the previously selected timing, following the same experimental procedure as described above. The data showed an insulin secretion peak after 15 minutes (p<0.0019), which, despite decreasing, confirmed to be significant also after 60 minutes (p<0.03) (FIG. 3, Panel B).

With the aim of better understanding the role of Prdx6 in the modulation of insulin secretion, the levels of cytoplasmic Ca²⁺ were measured to verify whether the secretion was Ca²⁺-dependent, similarly to what occurs after stimulation with glucose. The intracellular levels of calcium were measured both under basal conditions and in response to stimulation with 400 nM Prdx6 for 15 minutes; a significant reduction was observed in the intracellular levels of Ca²⁺ at the end of the treatment, indicating that Prdx6-mediated insulin secretion is a calcium-dependent process (FIG. 3, Panel C). In order to confirm that the mitochondrial defect observed in Prdx6^{KD} cells was a key mechanism for modifying the insulin secretion, the knockdown cells were also stimulated with recombinant Prdx6 (FIG. 3, Panel D). As expected, there being a morphological-functional defect of the mitochondria, no increase was observed in insulin secretion in response to the treatment with Prdx6. Since no increase in insulin secretion was found after stimulation with exogenous Prdx6 in the knockdown cells, it can be hypothesised that the effect of Prdx6 in stimulating insulin secretion is receptor-dependent and not mediated by the transport of Prdx6 into the cell. With the aim of confirming the hypothesis, the localisation of Prdx6 was analysed after stimulation with biotinylated recombinant Prdx6. In particular, using the EZ-Link NHS-Biotin Reagents kit (Invitrogen) and following the protocol provided, recombinant Prdx6 was biotinylated and used at a concentration of 400 nM to stimulate the cells for 15 minutes, as previously described. The cells thus treated were lysed on ice for 30 minutes with a lysis buffer containing 1% NP-40 (137 mM NaCI, 20 mM Tris pH 7.6, 1 mM MgCl₂, 1 mM CaCl₂, 10% Glycerol, protease inhibitor cocktail, phosphatase inhibitor cocktail). The lysate was centrifuged at 14,000 rpm - 18,600 g for 20 minutes at +4° C. and the supernatant with the cytoplasmic proteins was recovered. The pellet containing the membrane was washed with PBS 3 times to eliminate the cytoplasmic contaminant.

Subsequently, the pellet was resuspended in a lysis buffer containing 1.5% NP-40 and kept on ice for 30 minutes. The lysate obtained, containing the membrane proteins, was then processed in an ultracentrifuge at 50,000 rpm - 150,600 g for 1 hour at +4° C. The supernatant was recovered and the membrane and cytoplasmic proteins were quantified by means of the Bradford assay. 20 µg of proteins were separated by SDS-PAGE on a polyacrylamide gel at a 4-12% gradient and subsequently transferred onto a nitrocellulose membrane. The membrane was then incubated with an anti-biotin antibody. The results showed that the biotinylated protein is localised mainly at the level of the cytoplasmic membrane, supporting the hypothesis of a receptor-dependent mechanism of action (FIG. 4, Panel A). After the removal (stripping) of the previously used primary antibody, the same membrane was incubated with an anti-Prdx6 antibody capable of detecting both the endogenous protein and the biotinylated one. The results obtained confirmed an increase in Prdx6 in the cytoplasmic membrane (FIG. 4, Panel B). The data were validated by cytofluorometric analysis, in which the localisation of biotinylated Prdx6 was evaluated at both an extra- and intracellular level. For extracellular marking, the cells were plated and treated as previously described. Subsequently, the cells were detached using trypsin and washed with PBS and the pellet was incubated with a PBS solution containing streptavidin diluted 1:10 (which interacts directly with biotin) for 30 minutes in the dark and at room temperature. This was followed by washing with PBS and the subsequent cytofluorometric analysis. For intracellular marking, on the other hand, the cells were plated for 24 hours as described above, detached with trypsin, washed with PBS and subsequently permeabilised and fixed using the BD Fixation/Permeabilization Solution Kit (BD). Afterwards, they were stimulated with 400 nM of biotinylated Prdx6 for 15 minutes and at the end of the stimulation they were washed to eliminate the excess protein component, incubated with the solution of streptavidin and processed as described for the extracellular marking. The results confirmed that biotinylated Prdx6 is mainly localised in the cytoplasmic membrane (FIG. 4, Panel C) and only a small part is internalised (FIG. 4, Panel D), suggesting the presence of a receptor-dependent mechanism of action.

Finally, an assessment was made of the Prdx6-mediated insulin secretion in human pancreatic islets of healthy donors not suitable for transplantation (Approved by the Ethics and Scientific Committee of Niguarda Ca′ Granda Hospital in the meeting of 16.12.2009) obtained from Prof. Federico Bertuzzi, Niguarda Hospital, Milan, and through the purchase of material from the company Tebu-Bio (HUMAN ISLETS: Tebu-Bio SRL, Cat. 196HIR-IEQ-1000 Magenta, Milan, Italy).

The islets were maintained in a specific culture medium for 24 h after their arrival to enable their physiological recovery after transport. They were subsequently withdrawn using a stereomicroscope and transferred to a 24-well multiwell plate, where they were treated as previously described for the insulin secretion. The results (normalised for the number of islets in each well) showed that stimulation with Prdx6 (400 nM) was capable of inducing a significant increase in insulin secretion compared both to the control cells (p<0.0001) and to the cells treated with glucose (p<0.0033) (FIG. 5). Furthermore, although the data were not significant, an increasing trend in insulin secretion was observed following co-treatment with glucose and Prdx6, compared to the treatment with glucose alone, suggesting a synergic mechanism of secretion (FIG. 5).

### EXAMPLE 2. EXPERIMENT DESIGN AND RESULTS: PRDX6 AND GLP-1

In order to better characterise the hormone-like action of Prdx6, moreover, an assessment was made of its effect in modulating the secretion of GLP-1 (Glucagon-like Peptide-1), a peptide hormone of 37 amino acids synthesised and secreted in the gut by the L cells of the ileum and colon, with an increase in secretion in the postprandial period. GLP-1 increases insulin secretion in response to glucose and, in patients with DMT2, this action is altered due to a defect in the secretion of GLP-1. In particular, as the incretin release mechanism is similar to the glucose-mediated insulin release mechanism, it was verified whether Prdx6 may have a role in boosting insulin secretion also by acting on the secretion of GLP-1. Therefore, colorectal adenocarcinoma cells, NCI-H716, (ATCC-CCL-251 [H716], Manassas, Virginia, USA) were maintained in RPMI 1640 culture medium with 10% FBS and penicillin/streptomycin and subsequently stimulated with Prdx6. In particular, the cells were plated at a density of 1 x10⁶ overnight in 24-well multiwell plates, previously treated with basal membrane matrix (BMM) (BD Biosciences) to allow a better adhesion of the NCI-H716 and differentiation towards endocrine cell lines. The cells were then incubated with a specific buffer (138 mM NaCI, 4.5 mM KCI, 4.2 mM NaHCO₃, 1.2 mM NaH₂PO₄, 2.5 mM CaCl₂, 1.2 mM MgCl₂, 10 mM HEPES and 0.1% (wt/vol) BSA (pH = 7)) and treated with 25 mM glucose and 400 nM Prdx6.

At the end of the treatment, GLP-1 (the active isoforms GLP-1 (7-36) GLP-1 (7-37)) was measured at time 0 and after 15, 30, 60 and 120 minutes by means of the ELISA assay (Millipore) (FIG. 6, Panel A). Surprisingly, the treatment with Prdx6 increased the secretion of GLP-1 with a peak after 15 minutes (218.46±34.2) (FIG. 6, Panel B) and the co-treatment with glucose further significantly increased the secretion of GLP-1 after 15 minutes (286.1±31.04) (FIG. 6, Panel C). These data highlight the potential action of Prdx6 as a ‘hormone’ that stimulates the secretion of GLP-1. In order to confirm the data obtained on GLP-1 secretion in vitro, it was evaluated whether Prdx6^{-/-} mouse models, currently available for purchase from The Jackson Lab, (Sacramento, CA, USA, strain B6.129-Prdx6tm1 Abf/Mmjax, MMRRC (Stock No:43402-JAX-1-cysPrx KO) and kindly donated by Prof. Xiaosong Wang (The Jackson Laboratory), showed a defect in GLP-1 production in the postprandial period (fed state). As shown in FIG. 7, Panel A, the amount of secreted GLP-1 was lower in Prdx6^{-/-} mice than in Wild Type (WT) mice (p<0.005), in association with an increase in glycaemia (p<0.005), suggesting an alteration in the GLP-1-mediated insulin secretion process in Prdx6^{-/-} mice (FIG. 7, Panel B). In order to further confirm the data obtained, the Prdx6^{-/-} mice were subjected to oral administration of a glucose load (OGTT) by gavage (2 g/kg glucose after 16 hours of fasting). As shown in FIG. 7, Panel C, 15 minutes after stimulation with glucose the Prdx6^{-/-} mice showed a significant reduction in GLP-1 secretion compared to the WT animals (p<0.05). The data reported above point to a pharmacological role of Prdx6 as an insulin secretagogue agent, which would be able for the first time to increase insulin secretion with an anti-apoptotic effect (this effect is amply documented in the literature in relation to the role of Prdx6 as an antioxidant enzyme). Furthermore, it would be the first hypoglycaemic agent capable of simultaneously stimulating the secretion of insulin and of GLP-1, reducing the levels of oxidative stress.

1-17. (canceled)

18. A method of treating diabetes mellitus, the method comprising administering to a patient peroxiredoxin 6 or a synthetic analogue thereof.

19. The method of claim 18, wherein said diabetes mellitus is type 2 diabetes mellitus or type 1 diabetes mellitus.

20. The method of claim 18, wherein the patient is affected by obesity.

21. The method of claim 18, wherein said synthetic analogue of peroxiredoxin 6 is a synthetic analogue modified in one or more catalytic domains selected from the peroxidase site, phospholipase-A₂ site, lysophosphatidylcholine acyltransferase site, dimerization site and sumoylation site.

22. The method of claim 18, wherein said synthetic analogue of peroxiredoxin 6 is selected from the following mutant Prdx6s: mutant with the dimerization site inhibited, mutant with the phospholipase-A₂ site inhibited, mutant with the peroxidase site inhibited, and mutant with the sumoylation site inhibited.

23. The method of claim 22, wherein said mutant with the dimerization site inhibited is selected from L145E, L148E and L145E/L148E.

24. The method of claim 22, wherein said mutant with the phospholipase-A₂ site inhibited is selected from S32A or S32T.

25. The method of claim 22, wherein said mutant with the peroxidase site inhibited is C47S.

26. The method of claim 22, wherein said mutant with the sumoylation site inhibited is selected from K122R, K142R and K122R/K142R.

27. The method of claim 18, wherein said administering is of a pharmaceutical composition that comprises peroxiredoxin 6 and/or a synthetic analogue thereof as an active ingredient, together with one or more excipients and/or adjuvants.

28. The method of claim 27, wherein said composition further comprises a hypoglycaemic agent other than peroxiredoxin 6 or said synthetic analogue thereof.

29. The method of claim 28, wherein said hypoglycaemic agent is selected frommetformin, GLP-1 agonists, DPP-4 inhibitors, SGLT-2 inhibitors, human insulin and slow, rapid or ultra-rapid analogue insulin.

30. The method of claim 27, wherein said composition is in a form for subcutaneous administration.

31. The method of claim 18, wherein said administering is by subcutaneous administration.

32. The method of claim 18, wherein said peroxiredoxin 6 or said synthetic analogue thereof is administered in combination with at least one hypoglycaemic agent other than peroxiredoxin 6 or said synthetic analogue thereof.

33. The method of claim 32, wherein said at least one hypoglycaemic agent is selected from metformin, GLP-1 agonists, DPP-4 inhibitors, SGLT-2 inhibitors, human insulin and slow, rapid or ultra-rapid insulin analogue.

34. The method of claim 33, wherein said at least one hypoglycemic agent and said peroxiredoxin 6 or said synthetic analogue thereof are administered separately or sequentially.

35. The method of claim 32, wherein the patient is affected by obesity.

36. A method of treating obesity, the method comprising administering to a patient peroxiredoxin 6, a synthetic analogue thereof or a pharmaceutical composition comprising peroxiredoxin 6 and/or a synthetic analogue thereof as an active ingredient, together with one or more excipients and/or adjuvants, for use in therapy for obesity.

37. The method of claim 36, wherein said synthetic analogue thereof is a synthetic analogue modified in one or more catalytic domains selected from the peroxidase site, the phospholipase-A₂ site, the lysophosphatidylcholine acyltransferase site, the dimerization site and the sumoylation site.

38. The method of claim 37, wherein said synthetic analogue of peroxiredoxin 6 is selected from the following mutant Prdx6s: mutant with the dimerization site inhibited, mutant with the phospholipase-A₂ site inhibited, mutant with the peroxidase site inhibited, and mutant with the sumoylation site inhibited.

39. The method of claim 38, wherein said mutant with the dimerization site inhibited is selected from L145E, L148E and L145E/L148E.

40. The method of claim 38, wherein said mutant with the phospholipase-A₂ site inhibited is selected from S32A or S32T.

41. The method of claim 38, wherein said mutant with the peroxidase site inhibited is C47S.

42. The method of claim 38, wherein said mutant with the sumoylation site inhibited is selected from K122R, K142R and K122R/K142R.
